(19) United States
(12) Patent Application Publication
(10) Pub. No.: US 2024/0285491 A1
(43) Pub. Date: Aug. 29, 2024

(54) Transparent Water-In-Oil-Type Lip Cosmetic Composition

(71) Applicant: LG Household & Health Care Ltd., Seoul (KR)

(72) Inventors: Bo Mi Mi Kwon, Seoul (KR); Ji Young Kim, Seoul (KR); Khee Hwan Choi, Seoul (KR); Yeon Hee Kang, Seoul (KR)

(73) Assignee: LG Household & Health Care Ltd., Seoul (KR)

(21) Appl. No.: 18/695,532

(22) PCT Filed: Sep. 21, 2022
(86) PCT No.: PCT/KR2022/014106

§ 371 Date: Mar. 26, 2024

(30) Foreign Application Priority Data
Sep. 30, 2021 (KR) 10-2021-0129458

(51) Int. Cl. A61K 8/34; A61Q 1/04

(52) U.S. Cl. CPC A61K 8/342; A61Q 1/04; A61K 2800/262; A61K 2800/34; A61K 2800/43

(57) ABSTRACT

The present invention relates to a transparent water-in-oil-type lip cosmetic composition comprising 2-10 wt % of water, 28-55 wt % of glycerin, and an oil phase, with respect to the total weight of the composition, and 50 wt % or more of octyldodecanol with respect to the total weight of the oil phase. The composition of the present invention has transparent appearance, thus imparting an aesthetic effect to products and at the same time, can impart gloss when applied onto lips, and can provide moisturization through the supply of moisture, and long-lasting coloring effect.

### TECHNICAL FIELD

The present invention relates to a transparent water-in-oil lip cosmetic composition, and specifically, to a cosmetic composition including water, glycerin, and octyldodecanol.

### BACKGROUND ART

In the development of cosmetics, aesthetics as well as the texture and function of cosmetics are very important factors. As an example of differentiating the aesthetic elements of cosmetics, there are cosmetics having a transparent appearance. A transparent appearance imparts a clear and clean image or a luxurious and beautiful image to products and allows the unique properties of the product to stand out.

Meanwhile, in the development of lip cosmetics, a water-in-oil (W/O) emulsification technique is commonly used for lip cosmetics, especially, lip gloss emulsions, because the technique is capable of imparting glossiness and moisturization through moisture supply using oils having a high refractive index in an oil phase. Among the oils, octyldodecanol may be used as a raw material for increasing the glossiness of cosmetics due to having a high refractive index of 1.453, is less sticky than other oils having similar refractive indices, and may be suitable as a solvent of ethyl cellulose which is a thickener.

However, it is difficult for generally prepared W/O lip gloss emulsions to realize a transparent appearance because the water phase is dispersed as small particles in the oil phase, causing a clouding phenomenon.

As a conventional technique for implementing W/O emulsions having a transparent appearance, there is a method of matching the refractive indices of the oil phase and the water phase using a silicone oil. However, as awareness that silicone oils cause water pollution and are harmful to the skin increases, consumers' negative perception is increasing, and its unique silky and light feel makes it unsuitable for use in glossy lip gloss formulations.

As another technique, there is a microemulsion method. When this method is used, emulsion particles are finely broken down to 100 nm or less, and light is not scattered by the particles, and thus a transparent appearance can be realized. However, the method has disadvantages in that the use of special equipment such as a high-pressure emulsification device is required or the use of a large amount of emulsifier may cause skin irritation.

Therefore, there is a need to develop a water-in-oil lip cosmetic that exhibits excellent glossiness by including octyldodecanol and realizes a transparent appearance without using a silicone oil and a high-pressure emulsification device.

### DISCLOSURE

### Technical Problem

The present invention is directed to providing a water-in-oil lip composition having a transparent appearance and a composition that imparts improved moisturization and glossiness to the lip skin and exhibits a long-lasting coloring effect.

### Technical Solution

The present invention was completed by discovering that transparency could be secured by increasing a glycerin content while decreasing a water content by mixing a significant amount of octyldodecanol in an oil phase.

One aspect of the present invention provides a transparent water-in-oil lip cosmetic composition which includes, with respect to the total weight of the composition, 2 to 10 wt % of water, 28 to 55 wt % of glycerin, and an oil phase and includes octyldodecanol in an amount of 50 wt % or more with respect to the total weight of the oil phase.

Another aspect of the present invention provides a transparent water-in-oil lip cosmetic composition that has fluidity and is in the form of lip gloss.

Still another aspect of the present invention provides a method of preparing a transparent water-in-oil lip composition, which includes: preparing an oil phase including octyldodecanol in an amount of 50 wt % or more with respect to the total weight of the oil phase; and mixing the oil phase with 2 to 10 wt % of water and 28 to 55 wt % of glycerin with respect to the total weight of the composition.

### Advantageous Effects

A composition according to the present invention can impart an aesthetic effect to a product due to having a transparent appearance, impart glossiness when applied onto the lips, exhibit excellent formulation stability even when stored for a long period of time, and provide moisturization through moisture supply and long-lasting coloring effect.

### DESCRIPTION OF DRAWINGS

FIG. 1 is an image showing the appearance of samples of Examples 1 to 4 after 4 weeks have elapsed according to Experimental Example 1 of the present invention.

FIG. 2 is an image showing the appearance of samples of Comparative Examples 1 to 9 after 4 weeks have elapsed according to Experimental Example 1 of the present invention.

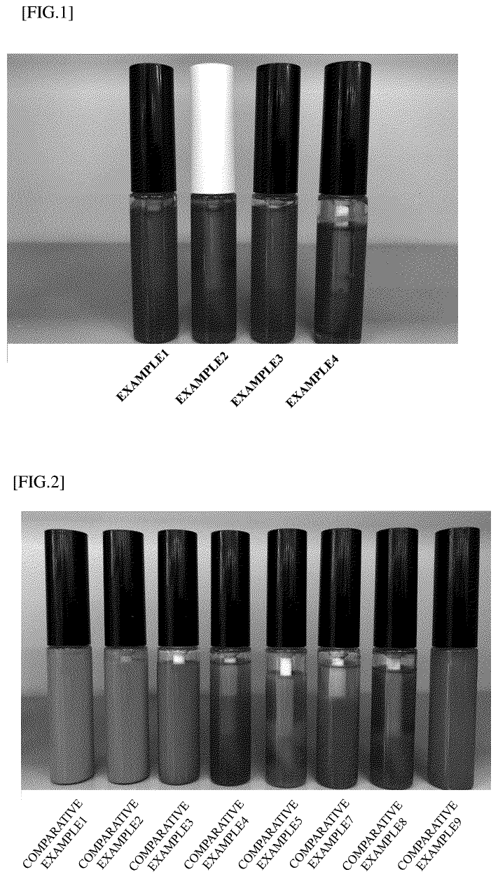

### MODES OF THE INVENTION

Hereinafter, the present invention will be described with reference to the accompanying drawings.

Meanwhile, each description and embodiment disclosed herein may also be applied to other descriptions and embodiments. That is, all combinations of the various elements disclosed herein fall within the scope of the present invention. In addition, the scope of the present invention is not limited by the specific description described below.

When a part is said to “include” a certain component, unless specified otherwise, this means that it may further include other components, rather than exclude other components.

The present invention refers to a transparent water-in-oil lip cosmetic composition, and the composition is characterized by including 50 wt % or more of octyldodecanol in an oil phase while including water and glycerin.

Specifically, the present invention provides a transparent water-in-oil lip cosmetic composition which includes, with respect to the total weight of the composition, 2 to 10 wt % of water, 28 to 55 wt % of glycerin, and an oil phase, and includes octyldodecanol in an amount of 50 wt % or more with respect to the total weight of the oil phase.

In the present invention, “transparency” refers to transmitting a part or all of the incident light, is a concept encompassing translucency, and can be interpreted to have the same meaning as typically understood by those skilled in the art. More specifically, “transparency” means that, when the composition of the present invention is placed in a transparent container, and a material smaller than the cross section of the transparent container is placed in the container, the material can be identified.

The composition of the present invention is characterized by having a transparent appearance even though it is an emulsified formulation. When applied onto the lips, the composition can impart glossiness and moisturization through moisture supply and maintain the color by coloring the stratum corneum of the lips.

The oil phase may be included in an amount of 30 to 70 wt % with respect to the total weight of the composition. Preferably, the content of the oil phase is 35 to 65 wt % or 37 to 63 wt %. The present invention relates to a water-in-oil cosmetic composition, and the composition may be formed by including a large amount of oil phase. The content of octyldodecanol may be determined based on the oil phase, and when octyldodecanol accounts for 50% or more of the oil phase, transparency may be exhibited by adjusting glycerin and water contents.

Octyldodecanol may be included in an amount of 50 wt % or more with respect to the total weight of the oil phase. Preferably, the content of octyldodecanol is 50 wt % to 90 wt %, and more preferably, 53 to 90 wt %, 50 to 85 wt %, or 53 to 85 wt %. Within the above range, a transparent appearance can be easily realized by adjusting water and glycerin contents, and when the content of octyldodecanol is less than 50 wt % with respect to the total weight of the oil phase, transparency may be degraded.

In an embodiment of the present invention, the octyldodecanol content may be 20 to 50 wt % with respect to the total weight of the composition. Preferably, the octyldodecanol content is 25 to 50 wt % or 25 to 45 wt %.

In the present invention, the content of water may be 2 wt % to 10 wt % with respect to the total weight of the composition. Preferably, the content is 2.5 to 10 wt %, 2 to 9.5 wt %, or 2.5 to 9.5 wt %, and most preferably, 3 to 9 wt %. When the water content is less than 2 wt %, there are limitations in dissolving dyes and salts, and thus preparation may not be possible, or the original function of a lip cosmetic composition to impart color may be lost. When the water content exceeds 10 wt %, a clouding phenomenon occurs, and thus it may be difficult to realize a transparent appearance.

In the present invention, the content of glycerin may be 28 to 55 wt % with respect to the total weight of the composition. Preferably, the content is 28 to 53 wt % or 30 to 50 wt %, and most preferably, 35 to 45 wt %. When the glycerin content is less than 28 wt %, transparency or moisture supply may be degraded, and adhesiveness may be degraded by the disappearance of a thickening ability due to emulsification. When the glycerin content exceeds 55 wt %, emulsification is not possible, or stability is poor, and thus commercialization may be difficult.

In the present invention, the oil phase may include one or more selected from the group consisting of oils, oil-phase thickeners, emollients, and emulsifiers in addition to octyldodecanol, but the present invention is not limited thereto. Any components that are used as an oil phase in the art may be used without limitation.

In an exemplary embodiment of the present invention, to increase the stability of the composition, the oil phase may include an emulsifier and an oil-phase thickener.

In an embodiment of the present invention, there is no particular limitation on the type of oil, any oil that is typically used in a cosmetic composition may be used, and examples of the oil include one or more selected from the group consisting of an ester-based oil, a hydrocarbon-based oil, a vegetable oil, and a silicone oil, but the present invention is not limited thereto.

As the ester-based oil, diisostearyl malate, cetyl ethylhexanoate, isocetyl ethylhexanoate, isocetyl myristate, isopropyl myristate, neopentyl glycol dicaprate, hexyl laurate, triethylhexanoin, pentaerythrityl tetraoctanoate, ethylhexyl palmitate, isononyl isononanoate, and the like may be exemplified. As the hydrocarbon-based oil, petroleum oils such as liquid paraffin, Vaseline, and isoparaffins, synthetic oils such as hydrogenated polydecene, synthetic squalane, and polybutene, and vegetable oils such as vegetable squalene may be exemplified. As the vegetable oil, olive oil, meadowfoam oil, rosehip oil, avocado oil, camellia oil, macadamia nut oil, castor oil, jojoba oil, and the like may be exemplified. As the silicone oil, dimethicone, cyclopentasiloxane, cyclohexasiloxane, methyl trimethicone, and the like may be exemplified, but the present invention is not limited thereto.

The oil may be included in an amount of 0.1 to 30 wt % with respect to the total weight of the composition. Preferably, the content of the oil is 0.5 to 15 wt % or 1 to 13 wt %.

In an embodiment of the present invention, the composition of the present invention may be substantially free of silicone oil. “Being substantially free of” may refer to being included in an amount of less than 0.5 wt %, less than 0.1 wt %, less than 0.001 wt %, or less than 0.00001 wt % with respect to the total weight of the composition.

In an embodiment of the present invention, the emulsifier may include one or more selected from silicone-based emulsifiers, stearate-based emulsifiers, and sorbitan-based emulsifiers, but the present invention is not limited thereto.

The silicone-based emulsifier may be one or more selected from the group consisting of cetyl PEG/PPG-10/1 dimethicone, PEG-10 dimethicone, lauryl PEG-9 polydimethylsiloxyethyl dimethicone, and lauryl polyglyceryl-3 polydimethylsiloxyethyl dimethicone.

The sorbitan-based emulsifier may be one or more selected from the group consisting of sorbitan olivate, sorbitan stearate, sorbitan isostearate, sorbitan tristearate, sorbitan sesquistearate, sorbitan palmitate, sorbitan laurate, sorbitan oleate, sorbitan sesquioleate, sorbitan trioleate, sorbitan stearate/sorbityl laurate, polysorbate 20, polysorbate 40, polysorbate 60, polysorbate 65, polysorbate 80, and polysorbate 85, but the present invention is not limited thereto.

The stearate-based emulsifier may be one or more selected from the group consisting of PEG-30 dipolyhydroxystearate, glyceryl stearate, PEG-100 stearate, triisostearate, and polyoxyethylene diisostearate, but the present invention is not limited thereto.

The silicone-based emulsifier may be included in an amount of 2 to 4 wt % with respect to the total weight of the composition. Preferably, the silicone-based emulsifier is included in an amount of 2 to 3.5 wt % or 2.5 to 3.5 wt %.

The stearate-based emulsifier may be included in an amount of 0.1 to 1 wt % with respect to the total weight of the composition. Preferably, the stearate-based emulsifier is included in an amount of 0.1 to 0.8 wt % or 0.3 to 0.8 wt %.

The sorbitan-based emulsifier may be included in an amount of 0.1 to 3 wt % with respect to the total weight of the composition. Preferably, the sorbitan-based emulsifier is included in an amount of 0.5 to 2 wt % or 0.8 to 1.5 wt %.

In an embodiment of the present invention, the emulsifier may include, with respect to the total weight of the composition, 2 to 4 wt % of the silicone-based emulsifier and 0.1 to 1 wt % of the stearate-based emulsifier.

In an embodiment of the present invention, the oil-phase thickener may be selected from the group consisting of: cellulose-based thickeners such as methyl cellulose, ethyl cellulose, cyclomethyl cellulose, and cycloethyl cellulose; dextrin-based or inulin-based thickeners such as dextrin palmitate/ethyl hexanoate, dextrin palmitate, and stearoyl inulin; vinylpyrrolidone-based polymers such as a VP/eicosene copolymer and a VP/hexadecene copolymer; copolymers such as an ethylenediamine/stearyl dimer dilinoleate copolymer and an ethylenediamine/hydrogenated dimer dilinoleate copolymer bis-di-C14-18 alkylamide; and fumed silica such as silica silylate and silica dimethyl silylate. It is preferable to use an oil-phase thickener that does not cause a clouding phenomenon after increasing viscosity, and the oil-phase thickener may be, for example, ethyl cellulose, but the present invention is not limited thereto.

The oil-phase thickener may be included in an amount of 0.5 to 3 wt % with respect to the total weight of the composition. Preferably, the oil-phase thickener is included in an amount of 1 to 2.5 wt % or 1.5 to 2.5 wt %.

The composition of the present invention may further include a viscosity adjusting agent, a coloring agent, a moisturizing agent, and the like, but the present invention is not limited thereto.

The composition of the present invention may further include 0.1 to 1 wt % of a pigment. The pigment is used to impart a coloring effect, and it is preferable to use a dye to realize a transparent appearance. When the content of the pigment is less than 0.1 wt %, the coloring effect may be insignificant, and when the content of the pigment exceeds 1 wt %, solubility may be problematic.

In an embodiment of the present invention, the composition of the present invention may include, with respect to the total weight of the composition, 2 to 4 wt % of the silicone-based emulsifier, 0.1 to 1 wt % of the stearate-based emulsifier, 0.1 to 1 wt % of the pigment, and 0.5 to 3 wt % of the oil-phase thickener.

In an embodiment of the present invention, the composition of the present invention exhibits fluidity, and the viscosity of the composition may range from 3000 to 20000 cps. The viscosity of the composition may be measured after filling a 100 ml stability container up to the neckline and then storing the container in a 25° C. constant-temperature tank for 1 day. Also, the viscosity may be measured at 30 rpm/min using spindle No. 4 in a Brookfield rheometer.

The composition of the present invention may maintain a liquid form by exhibiting fluidity at room temperature and may be stored in various types of containers.

The lip cosmetic composition of the present invention may be a liquid composition and may be prepared in the form of lip gloss or lip liquid. Preferably, the lip cosmetic composition may be prepared in the form of lip gloss.

The cosmetic composition of the present invention may be used according to a typical method of use, and the frequency of use may vary depending on the skin condition or preference of a user.

The above-described components included in the cosmetic composition according to the present invention may be included in the cosmetic composition of the present invention within a range not exceeding the maximum amount specified in standards related to “Cosmetic Use and Permission” prescribed by each government.

In addition, the present invention may relate to a use of the cosmetic composition to manufacture a cosmetic. The description of the cosmetic composition of the present invention for manufacturing a cosmetic may be the same as described above for the cosmetic composition.

The present invention also provides a method of preparing the transparent water-in-oil lip composition according to the present invention.

The preparation method of the present invention includes: preparing an oil phase including octyldodecanol in an amount of 50 wt % or more with respect to the total weight of the oil phase; and mixing the oil phase with 2 to 10 wt % of water and 28 to 55 wt % of glycerin with respect to the total weight of the composition.

In the preparation method, the oil phase may include one or more selected from the group consisting of oils, oil-phase thickeners, emollients, and emulsifiers.

In the preparation method, the emulsifier may include, with respect to the total weight of the composition, 2 to 4 wt % of a silicone-based emulsifier and 0.1 to 1 wt % of a stearate-based emulsifier.

In the preparation method, the mixing with water and glycerin may be a step of mixing the oil phase sequentially with water and glycerin.

The descriptions of the oil phase, octyldodecanol, water, glycerin, and cosmetic composition may be the same as described above for the cosmetic composition.

Hereinafter, the present invention will be described in detail with reference to the following experimental examples. However, it should be understood that the following experimental examples are given for the purpose of illustration only and are not intended to limit the scope of the present invention. In addition, these experimental examples are only intended to aid in understanding the present invention, and the scope of the present invention is not limited to the following experimental examples.

### EXAMPLES

### Preparation Example 1. Preparation of Composition

The following Components 1 to 6 were mixed at room temperature in a first agi-mixer, then heated to 90° C., and stirred using the agi-mixer. Components 7, 9, and 10 were mixed at 40° C. in a second agi-mixer and then stirred, and Component 8 was added thereto and then stirred using the agi-mixer. The two agi-mixers were stabilized at 60° C., and the mixture in the second agi-mixer was added to the mixture in the first agi-mixer, stirred using a homo mixer for 10 minutes, and cooled to 28° C. As a result, compositions of Examples 1 to 4 and Comparative Examples 1 to 9, which have the compositions shown in the following Tables 1 and 2, were prepared.

TABLE 1

| | Components | Use | Example 1 | Example 2 | Example 3 | Example 4 |
|---|---|---|---|---|---|---|
| 1 | Octyldodecanol | Emollient | 42.5 | 42.5 | 30.0 | 30.0 |
| 2 | Ethyl cellulose | Oil-phase thickener | 2.0 | 2.0 | 2.0 | 2.0 |
| 3 | Diisostearyl malate | Emollient | to 100 | to 100 | to 100 | to 100 |
| 4 | Cetyl PEG/PPG-10/1 dimethicone | Emulsifier | 2.7 | 2.7 | 2.7 | 2.7 |
| 5 | Sorbitan sesquioleate | Emulsifier | 1.0 | 1.0 | 1.0 | 1.0 |
| 6 | PEG-30 dipolyhydroxystearate | Emulsifier | 0.5 | 0.5 | 0.5 | 0.5 |
| 7 | Sodium chloride | Viscosity adjusting agent | 0.3 | 0.3 | 0.3 | 0.3 |
| 8 | Glycerin | Moisturizing agent | 30.0 | 40.0 | 40.0 | 50.0 |
| 9 | Pigment | Coloring agent | 0.4 | 0.4 | 0.4 | 0.4 |
| 10 | water | water | 9.0 | 9.0 | 3.0 | 9.0 |

| | | | | | |
|---|---|---|---|---|---|
| 11 | Ratio of octyldodecanol in oil phase (Components 1 to 6) (wt %) | 70.5 | 84.5 | 53.3 | 74.4 |

TABLE 2

| | | | Compar- | Compar- | Compar- | Compar- | Compar- | Compar- | Compar- | Compar- | Compar- |
|---|---|---|---|---|---|---|---|---|---|---|---|
| | | | ative | ative | ative | ative | ative | ative | ative | ative | ative |
| | | | Exam- | Exam- | Exam | Exam- | Exam- | Exam- | Exam- | Exam- | Exam- |
| | Components | Use | ple 1 | ple 2 | ple 3 | ple 4 | ple 5 | ple 6 | ple 7 | ple 8 | ple 9 |
| 1 | Octyldodecanol | Emollient | 10 | 42.5 | 42.5 | 50.0 | 72.0 | 30.0 | 10.0 | 81.0 | 30.0 |
| 2 | Ethyl cellulose | Oil-phase | 2.0 | 2.0 | 2.0 | 3.0 | 2.0 | 2.0 | 2.0 | 2.0 | 2.0 |
| | | thickener | | | | | | | | | |
| 3 | Diisostearyl malate | Emollient | to 100 | to 100 | to 100 | to 100 | to 100 | to 100 | to 100 | to 100 | to 100 |
| 4 | Cetyl PEG/PPG-10/1 | Emulsifier | 2.7 | 2.7 | 2.7 | 2.7 | 2.7 | 2.7 | 2.7 | 2.7 | 2.7 |
| | dimethicone | | | | | | | | | | |
| 5 | Sorbitan sesquioleate | Emulsifier | 1.0 | 1.0 | 1.0 | 1.0 | 1.0 | 1.0 | 1.0 | 1.0 | 1.0 |
| 6 | PEG-30 | Emulsifier | 0.5 | 0.5 | 0.5 | 0.5 | 0.5 | 0.5 | 0.5 | 0.5 | 0.5 |
| | dipolyhydroxystearate | | | | | | | | | | |
| 7 | Sodium chloride | Viscosity | 0.3 | 0.3 | 0.3 | 0.3 | 0.3 | 0.3 | 0.3 | 0.3 | 0.3 |
| | | adjusting | | | | | | | | | |
| | | agent | | | | | | | | | |
| 8 | Glycerin | Moisturizing | 12.0 | 12.0 | 30.0 | 25.0 | 10.0 | 40.0 | 30.0 | 9.0 | 60.0 |
| | | agent | | | | | | | | | |
| 9 | Pigment | Coloring | 0.4 | 0.4 | 0.4 | 0.4 | 0.4 | 0.4 | 0.4 | 0.4 | 0.4 |
| | | agent | | | | | | | | | |
| 10 | water | water | 30.0 | 30.0 | 20.0 | 8.0 | 3.0 | 1.0 | 9.0 | 3.0 | 3.0 |

| | | | | | | | | | | |
|---|---|---|---|---|---|---|---|---|---|---|
| 11 | Ratio of octyldodecanol | 17.5 | 74.2 | 86.2 | 75.4 | 83.4 | 51.5 | 16.6 | 92.8 | 82.6 |
| | in oil phase (Components | | | | | | | | | |
| | 1 to 6) (wt %) | | | | | | | | | |

### Experimental Example 1. Evaluation of Transparency, Stability, and Adhesiveness

The transparency, stability, and adhesiveness of the compositions of Examples 1 to 4 and Comparative Examples 1 to 9, which were prepared according to Preparation Example 1, were evaluated.

Transparency was evaluated according to the result of visual observation immediately after the composition was prepared, and stability was evaluated according to whether the composition separated at room temperature after 4 weeks. The degree of adhesiveness was evaluated by conducting a blind test with 10 women in their 20s to 30s, and results thereof were summarized.

TABLE 3

| Evaluation items | Example 1 | Example 2 | Example 3 | Example 4 |
|---|---|---|---|---|
| Transparency | ◯ | ⊚ | ⊚ | ◯ |
| Stability (no separation) | ◯ | ◯ | ◯ | ◯ |
| Adhesiveness | ◯ | ◯ | ◯ | ◯ |

TABLE 4

| | Compar- | Compar- | Compar- | Compar- | Compar- | Compar- | Compar- | Compar- | Compar- |
|---|---|---|---|---|---|---|---|---|---|
| | ative | ative | ative | ative | ative | ative | ative | ative | ative |
| Evaluation | Exam- | Exam- | Exam- | Exam- | Exam- | Exam- | Exam- | Exam- | Exam- |
| items | ple 1 | ple 2 | ple 3 | ple 4 | ple 5 | ple 6 | ple 7 | ple 8 | ple 9 |
| Transparency | X | X | X | Δ | ◯ | — | Δ | ⊚ | X |
| Stability | ◯ | ◯ | ◯ | X | X | X | X | X | X |
| (no separation) | | | | | | | | | |
| Adhesiveness | ◯ | ◯ | ◯ | ◯ | X | — | ◯ | X | ◯ |

⊚: very good
◯: good
Δ: fair
X: poor
—: could not be evaluated

As shown in Table 3, in the case of all of Examples 1 to 4 in which octyldodecanol, water, and glycerin contents were within the ranges of the present invention, transparency, stability, and adhesiveness were excellent.

As shown in Table 4, in the case of Comparative Examples 1 to 3 in which water contents were 30 wt %, 30 wt %, and 20 wt %, respectively, transparency was degraded. In the case of Comparative Example 6 in which a water content was 1 wt %, since the pigment and salt did not dissolve and an emulsion was not produced, the evaluation of transparency and adhesiveness was not possible, and stability was degraded. In the case of Comparative Example 7 in which an octyldodecanol content was less than 50 wt % with respect to the total content of the oil phase, both transparency and stability were degraded. In the case of Comparative Examples 4, 5, and 8 in which glycerin contents were 25 wt %, 10 wt %, and 9 wt %, respectively, emulsion particles were not properly formed due to an insufficient water phase content, and thus stability was degraded, and adhesiveness was also degraded. Also, in the case of Comparative Example 9 in which a glycerin content was 60 wt %, transparency and stability were degraded.

### Experimental Example 2. Evaluation of Moisturization and Glossiness

The moisturization through moisture supply and glossiness of the compositions of Example 4 and Comparative Examples 1 and 8, which were prepared according to Preparation Example 1, were evaluated.

Moisturization through moisture supply was measured using a skin hydration level analyzer (Corneometer) and evaluated as ⊚ when skin moisture retention was increased 100% or more 2 hours after application, o when skin moisture retention was increased 30 to 100%, Δ when skin moisture retention was increased 0 to 30%, and X when skin moisture retention was decreased.

Glossiness was measured using a glossmeter (incident angle: 75°) after the sample was applied onto black paper to a thickness of 0.3 mm, and indicated as ⊚ when glossiness was 90 or more, o when glossiness was less than 90 and 70 or more, and Δ when glossiness was less than 70 and 50 or more.

TABLE 5

| | | Comparative | Comparative |
|---|---|---|---|
| Evaluation items | Example 4 | Example 1 | Example 8 |
| Moisturization | ⊚ | Δ | Δ |
| through water supply | | | |

TABLE 6

| Evaluation items | Example 4 | Comparative Example 1 |
|---|---|---|
| Glossiness | ⊚ | Δ |

As shown in Tables 5 and 6, the skin moisture retention and glossiness of the composition of Example 4 were significantly improved, whereas the skin moisture retention of the compositions of Comparative Examples 1 and 8 was low, and the glossiness of Comparative Example 1 was degraded.

1. A transparent water-in-oil lip cosmetic composition comprising, with respect to a total weight of the composition, 2 to 10 wt % of water, 28 to 55 wt % of glycerin, and an oil phase and comprising octyldodecanol in an amount of 50 wt % or more with respect to a total weight of the oil phase.

2. The composition of claim 1, wherein the oil phase is included in an amount of 30 to 70 wt % with respect to the total weight of the composition.

3. The composition of claim 1, wherein octyldodecanol is included in an amount of 50 wt % to 90 wt % with respect to the total weight of the oil phase.

4. The composition of claim 1, wherein the oil phase includes one or more selected from the group consisting of an oil, an oil-phase thickener, an emollient, and an emulsifier in addition to octyldodecanol.

5. The composition of claim 4, wherein the emulsifier includes, with respect to the total weight of the composition, 2 to 4 wt % of a silicone-based emulsifier and 0.1 to 1 wt % of a stearate-based emulsifier.

6. The composition of claim 1, further comprising 0.1 to 1 wt % of a pigment.

7. English Specification The composition of claim 1, wherein the composition is in the form of lip gloss.

8. A method of preparing a transparent water-in-oil lip composition, comprising:
preparing an oil phase including octyldodecanol in an amount of 50 wt % or more with respect to a total weight of the oil phase; and
mixing the oil phase with 2 to 10 wt % of water and 28 to 55 wt % of glycerin with respect to a total weight of the composition.

9. The method of claim 8, wherein the oil phase includes one or more selected from the group consisting of an oil, an oil-phase thickener, an emollient, and an emulsifier.

10. The method of claim 9, wherein the emulsifier includes, with respect to the total weight of the composition, 2 to 4 wt % of a silicone-based emulsifier and 0.1 to 1 wt % of a stearate-based emulsifier.
